(19) United States
(12) Patent Application Publication
(10) Pub. No.: US 2009/0143609 A1
(43) Pub. Date: Jun. 4, 2009

(54) LIQUID CRYSTAL COMPOSITIONS AND THEIR USE IN LIQUID CRYSTAL DEVICES

(76) Inventors: Kotaro Araya, Hitachiota (JP); Shintaro Takeda, Hitachi (JP); Hiroyuki Kagawa, Hitachinaka (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/324,921

(22) Filed: Nov. 28, 2008

(30) Foreign Application Priority Data
Nov. 30, 2007 (JP) 2007-309639

(51) Int. Cl. C07C 15/12; C07C 43/20; C07C 69/76

(52) U.S. Cl. 560/73; 568/642; 585/25

(57) ABSTRACT

To provide a liquid crystal composition having low viscosity in a wide temperature range and to provide a liquid crystal device excellent in response speed in the wide temperature range, a compound represented by the following general formula (1) or (2) is contained in the composition.

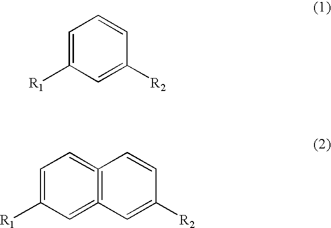

In the general formulae (1) and (2), R₁ and R₂ are each independently selected from alkyl groups, alkoxy groups, F atom, Cl atom, OCF₃, CF₃, phenyl groups each having a substituent, and phenyl rings each connected with a bridging group and having a substituent.

### CROSS-REFERENCE TO RELATED APPLICATION

The present application claims priority from Japanese application JP 2007-309639 filed on Nov. 30, 2007, the content of which is hereby incorporated by reference into this application.

### BACKGROUND OF THE INVENTION

1. Field of the Invention

The present invention relates to a nematic liquid crystal composition and a liquid crystal device using the same.

2. Description of the Related Art

Liquid crystal devices are already known as devices which include two glass substrates and a liquid crystal layer disposed therebetween and display letters, numbers, figures, pictures, and the like owing to electro-optical effects. At present, there are known various driving modes such as a twisted nematic (TN) mode, a super twisted nematic (STN) mode, an in-plane switching (IPS) mode, a vertical alignment (VA) mode, an optically compensated birefringence (OCB) mode. Recently, the liquid crystal devices using those driving modes are manufactured for use not only as a liquid crystal device for a desktop personal computer but also as a large-screen liquid crystal device for a liquid crystal television, whereas development on a compact liquid crystal device such as a cellular phone has also been rapid.

From a viewpoint that the liquid crystal device is expected to be used as a display device for the liquid crystal television, speeding-up in response is required for displaying moving images in sport programs and other similar programs. A liquid crystal composition meeting this demand is required to have lower viscosity compared with a conventional liquid crystal composition. In accordance with such a trend described above, fluorine nematic liquid crystal compositions have been developed in place of cyano nematic liquid crystal compositions used in STN liquid crystal (“Chemistry of Liquid Crystal,” Japan Scientific Societies Press, 1994, pp. 40-49). Compared with the cyano nematic liquid crystal compositions, those fluorine nematic liquid crystal compositions are high in specific resistance, which cause less unevenness in displayed images to be considered as liquid crystal compositions having high reliability.

### SUMMARY OF THE INVENTION

As described above, the large-screen liquid crystal device is manufactured for the liquid crystal television, whereas the development on the compact liquid crystal device such as a cellular phone has also been rapid. However, while a liquid crystal composition which accommodates to the liquid crystal television has been developed, development on a liquid crystal composition for the compact liquid crystal device such as a cellular phone has not seen much progress. This is because an environment in which the liquid crystal device is used is different between the former case and the latter case. That is, this is because, while the liquid crystal television is used for viewing at temperatures in the vicinity of room temperature, the compact liquid crystal device such as a cellular phone is used in a wide temperature range including a cold environment in winter and a hot environment in summer.

Under those circumstances, the development on the liquid crystal compositions has focused on accommodation to an operating environment at temperatures in the vicinity of room temperature, and hence it seems that not so much attention has been directed to an operating environment having the wide temperature range. From a viewpoint of properties of the liquid crystal compositions, conventional approaches have focused attention on viscosity and response speed at room temperature. It can be said that, with regard to the compact liquid crystal device such as a cellular phone, attention must be paid on viscosity and response speed in a wide temperature range.

Therefore, it is an object of the present invention to provide a liquid crystal composition having low viscosity in a wide temperature range and to provide a liquid crystal device excellent in response speed in the wide temperature range.

With a view to solving the above-mentioned problems, intensive studies have been made on a liquid crystal composition having a low viscosity in a wide temperature range, that is, a liquid crystal device excellent in response speed in a wide temperature range, and thus the present invention has been completed.

According to a first aspect of the present invention, there is used, as means for solving the problems, a liquid crystal composition containing a compound represented by the following general formula (1),

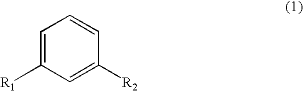

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

According to a second aspect of the present invention, there is used, as means for solving the problems, a liquid crystal composition containing a compound represented by the following general formula (2),

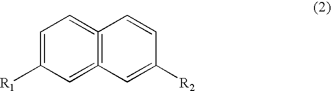

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

According to a third aspect of the present invention, there is used, as means for solving the problems, a liquid crystal composition containing a compound represented by the following general formula (3),

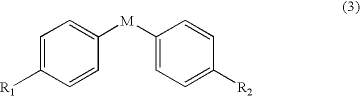

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent; and M is selected from CH₂, O, NH, and C═O.

According to a fourth aspect of the present invention, there is used, as means for solving the problems, a liquid crystal composition according to any one of the first, second and third aspects of the invention, containing a compound represented by the following general formula (4),

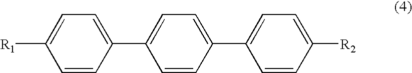

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

According to a fifth aspect of the present invention, there is used, as means for solving the problems, a liquid crystal composition according to any one of the first, second and third aspects of the invention, containing a compound represented by the following general formula (5),

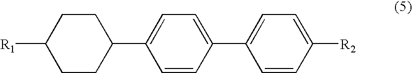

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

According to a sixth aspect of the present invention, there is used, as means for solving the problems, a liquid crystal composition according to any one of the first, second and third aspects of the invention, containing a compound represented by the following general formula (6),

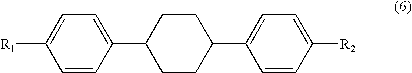

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

According to a seventh aspect of the present invention, there is used, as means for solving the problems, a liquid crystal composition according to any one of the first, second and third aspects of the invention, containing a compound represented by the following general formula (7),

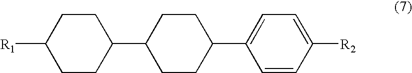

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

According to an eighth aspect of the present invention, there is used, as means for solving the problems, a liquid crystal composition according to any one of the first, second and third aspects of the invention, containing a compound represented by the following general formula (8),

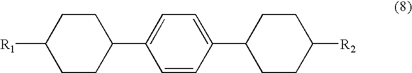

where: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

According to a ninth aspect of the present invention, there is used, as means for solving the problems, a liquid crystal composition including a compound containing, in the same compound, two or more linking groups out of linking groups represented by the following general formulae (9), (10), and (11),

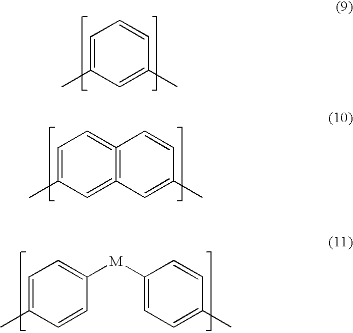

where M is selected from CH₂, O, NH, and C═O in the general formula (11).

According to a tenth aspect of the present invention, there is used, as means for solving the problems, a liquid crystal device including the compound represented by the general formula (1) in a liquid crystal composition. According to an eleventh aspect of the present invention, there is used, as means for solving the problems, a liquid crystal device including the compound represented by the general formula (2) in a liquid crystal composition. According to a twelfth aspect of the present invention, there is used, as means for solving the problems, a liquid crystal device including the compound represented by the general formula (3) in a liquid crystal composition.

According to a thirteenth aspect of the present invention, there is used, as means for solving the problems, a liquid crystal device according to anyone of the tenth, eleventh and twelfth aspects of the invention, including the compound represented by the general formula (4) in a liquid crystal composition. According to a fourteenth aspect of the present invention, there is used, as means for solving the problems, a liquid crystal device according to any one of the tenth, eleventh and twelfth aspects of the invention, including the compound represented by the general formula (5) in a liquid crystal composition. According to a fifteenth aspect of the present invention, there is used, as means for solving the problems, a liquid crystal device according to any one of the tenth, eleventh and twelfth aspects of the invention, including the compound represented by the general formula (6) in a liquid crystal composition. According to a sixteenth aspect of the present invention, there is used, as means for solving the problems, a liquid crystal device according to any one of the tenth, eleventh and twelfth aspects of the invention, including the compound represented by the general formula (7) in a liquid crystal composition. According to a seventeenth aspect of the present invention, there is used, as means for solving the problems, a liquid crystal device according to any one of the tenth, eleventh and twelfth aspects of the invention, including the compound represented by the general formula (8) in a liquid crystal composition.

According to an eighteenth aspect of the present invention, there is used, as means for solving the problems, a liquid crystal device including, in a crystal liquid composition, a compound containing two or more linking groups out of the linking groups represented by the general formulae (9), (10), and (11) in the same compound.

As described above, by using a nematic liquid crystal composition of the present invention, a liquid crystal composition having a low viscosity in a wide temperature range can be provided, and also, a liquid crystal device excellent in response speed in a wide temperature range can be provided.

### BRIEF DESCRIPTION OF THE DRAWING

FIG. 1 is an explanatory view showing a liquid crystal filling process of a liquid crystal device according to the present invention.

### DETAILED DESCRIPTION OF THE INVENTION

Hereinafter, the liquid crystal composition according to the present invention and the liquid crystal device using the composition are described in detail.

A phenyl group having a substituent according to the present invention is represented by the following general formula (12),

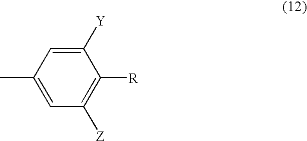

where R is selected from alkyl groups, alkoxy groups, F atom, Cl atom, OCF₃, and CF₃; Y is chosen from H atom and F atom; and Z is also chosen from H atom and F atom.

A linking group according to the present invention is, in a restricted sense, represented by M in the following general formula (13),

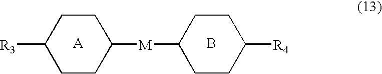

where R₃ and R₄ are each independently selected from alkyl groups, alkoxy groups, F atom, Cl atom, OCF₃, CF₃, phenyl groups each having a substituent, phenyl rings each connected with a bridging group and having a substituent; A and B are each independently selected from a cyclohexane ring and a phenyl ring,

and the linking group has the following chemical structure: —CH═N—, —C(═O)—O—, —N═N—, —CH═CH—, —CH₂—O—, —CH₂—CH₂—, —CF₂—O—

and in a broad sense, the linking group includes M in the following general formulae (14) and (15),

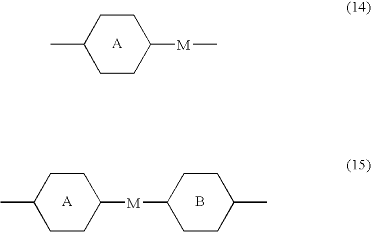

As a compound according to the present invention containing two or more linking groups out of those represented by general formulae (9), (10), and (11) in the same compound, a compound represented by the following general formula (16) is exemplified,

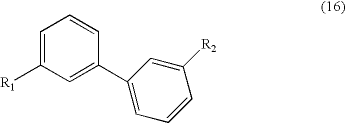

where R₁ and R₂ are each independently selected from alkyl groups, alkoxy groups, F atom, Cl atom, OCF₃, CF₃, phenyl groups each having a substituent, and phenyl rings each connected with a bridging group and having a substituent.

In the compound represented by the general formula (16), two linking groups are incorporated in the same compound, and respective linking groups are arranged in an overlapped manner.

A compound represented by the following general formula (17) is also one of the examples. In the compound represented by the general formula (17), two linking groups are incorporated in the same compound, and respective linking groups are independently arranged without being overlapped.

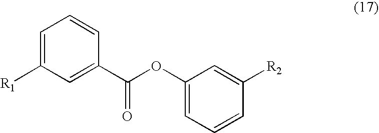

where R₁ and R₂ are each independently selected from alkyl groups, alkoxy groups, F atom, Cl atom, OCF₃, CF₃, phenyl groups each having a substituent, and phenyl rings each connected with a bridging group and having a substituent.

Next, embodiments of the liquid crystal device according to the present invention using a nematic liquid crystal composition and a production method therefor are described in detail.

### Embodiment 1

Table 1 shows property values of ZLI-1132 (manufactured by Merck KGaA), which is a standard liquid crystal composition as a host liquid crystal of the present invention. For measuring clearing points (Tni), a differential scanning calorimeter (DSC) and a polarizing microscope were used. For measuring viscosities, a rotating viscometer was used.

TABLE 1

| | | HOST | | | | |
|---|---|---|---|---|---|---|
| | | LIQUID | | | VISCOSITY | VISCOSITY |
| | No | CRYSTAL | DOPANT | Tni(° C.) | (25° C.) | (5° C.) |
| EXAMPLE 1 | 1 | ZLI1132 | NONE | 70 | 21 | 70 |
| EXAMPLE 1 | 2 | ZLI1132 | 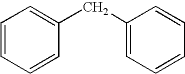 | 60 | 18 | 51 |
| EXAMPLE 2 | 3 | ZLI1132 | 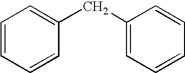 | 70 | 20 | 60 |
| | | | 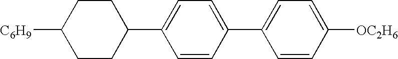 | | | |
| EXAMPLE 3 | 4 | ZLI1132 | 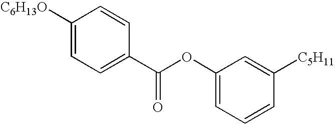 | 67 | 20 | 59 |
| EXAMPLE 4 | 5 | ZLI1132 | 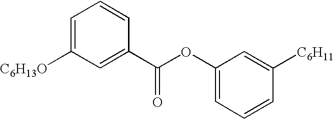 | 64 | 20 | 55 |
| COMPARATIVE EXAMPLE 1 | 6 | ZLI1132 | 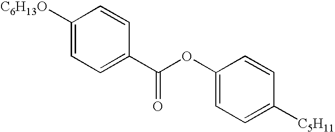 | 70 | 23 | 80 |

5 wt % diphenyl methane represented by a molecular structure (18) was added as a dopant to ZLI-1132, whereby Liquid Crystal Composition EXAMPLE 1 was obtained.

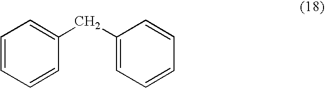

No clearing point for diphenyl methane was observed. Table 1 shows property values of Liquid Crystal Composition EXAMPLE 1. As is clear from Table 1, decrease in the clearing point due to addition of diphenyl methane was observed, but large decrease was not observed. In particular, attention is focused on remarkable decrease in viscosity at 5°C. A reason thereof is not clear. However, diphenyl methane does not show a linear structure as found in conventional liquid crystal compounds, and therefore, the bent core structure may effectively affect the decrease in the viscosity. With this in mind, diphenyl ether showing a similar structure was added to ZLI-1132 and researched, whereby almost the same results as in diphenyl methane were obtained. Accordingly, addition of a compound having a bent core structure maybe effective for the decrease in the viscosity at a low temperature side. The addition is also effective for the decrease in the viscosity in an entire temperature region of an operating temperature range of the liquid crystal device.

### Embodiment 2

In this embodiment, 5 wt % diphenyl methane as a dopant was added to ZLI-1132 (manufactured by Merck KGaA). Further, 5 wt % 1-ethoxy-4′-hexylcyclohexyl-biphenyl represented by a molecular structure (19) was added, whereby Liquid Crystal Composition EXAMPLE 2 was obtained.

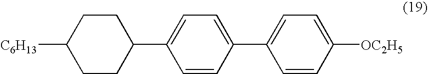

A clearing point for 1-ethoxy-4′-hexylcyclohexyl-biphenyl was observed at 148° C. Table 1 shows property values of Liquid Crystal Composition EXAMPLE 2.

As is clear from Table 1, there is no decrease in the clearing point due to addition of diphenyl methane, and only decrease in the viscosity at 5° C. was observed. This is because the decrease in the clearing point associated with the addition of diphenyl methane was compensated with increase in the clearing point associated with the addition of 1-ethoxy-4′-hexylcyclohexyl-biphenyl. As is clear from this embodiment, in order to decrease the viscosity at a low temperature side without decrease in the clearing point, addition of a liquid crystal compound having a high clearing point as well as a compound having a bent core structure may be effective.

### Embodiment 3

In this embodiment, 5 wt % 4-hexyloxylbenzoic-3′-pentyl-phenylester represented by a molecular structure (20) was added as a dopant to ZLI-1132 (manufactured by Merck KGaA), whereby Liquid Crystal Composition EXAMPLE 3 was obtained.

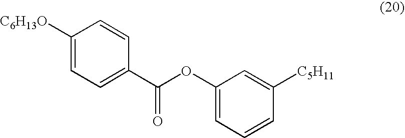

No clearing point for 4-hexyloxylbenzoic-3′-pentyl-phenylester was observed. Table 1 shows property values of this novel Liquid Crystal Composition EXAMPLE 3.

Unlike the case of diphenyl methane shown in Example 1, large decrease in viscosity at 5° C. could be observed in spite of small decrease in the clearing point. The 4-hexyloxylbenzoic-3′-pentyl-phenylester has a bent core structure due to incorporation of an alkyl chain into a meta, and the structure may contribute to the decrease in the viscosity. On the other hand, a length of a molecule of 4-hexyloxylbenzoic-3′-pentyl-phenylester is larger than that of diphenyl methane, which may lessen the decrease in the clearing point. In the foregoing, by introducing both points of the bent core structure and molecular length into the molecular structure, it was possible to bring out property values such as the decrease in the viscosity at low temperatures with no decrease in the clearing point.

### Embodiment 4

In this embodiment, 5 wt % 3-hexyloxylbenzoic-3′-pentyl-phenylester represented by a molecular structure (21) was added as a dopant to ZLI-1132 (manufactured by Merck KGaA), whereby Liquid Crystal Composition EXAMPLE 4 was obtained.

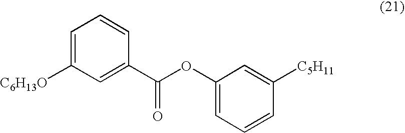

No clearing point for 3-hexyloxylbenzoic-3′-pentyl-phenylester was observed. Table 1 shows property values of Liquid Crystal Composition EXAMPLE 4.

Unlike the case of diphenyl methane shown in Example 1, large decrease in viscosity at 5° C. could be observed in spite of small decrease in the clearing point. The 3-hexyloxylbenzoic-3′-pentyl-phenylester has two bent core structures due to incorporation of an alkyl chain or the like into two metas, and the structure may contribute to the decrease in the viscosity. On the other hand, a length of a molecule of 3-hexyloxylbenzoic-3′-pentyl-phenylester is larger than that of diphenyl methane, which may lessen the decrease in the clearing point. In the foregoing, by introducing both points of the bent core structure and molecular length into the molecular structure, it was possible to bring out property values such as the decrease in the viscosity at low temperatures with no decrease in the clearing point.

### COMPARATIVE EXAMPLE 1

In this comparative example, 5 wt % 4-hexyloxylbenzoic-4′-pentyl-phenylester represented by a molecular structure (22) was added as a dopant to ZLI-1132 (manufactured by Merck KGaA), whereby Liquid Crystal Composition Comparative Example 1 was obtained.

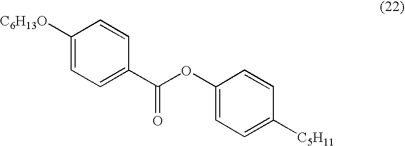

A clearing point could be observed for 4-hexyloxylbenzoic-4′-pentyl-phenylester at 70° C. Table 1 shows property values of this novel Liquid Crystal Composition Comparative Example 1.

Unlike the case of 4-hexyloxylbenzoic-4′-pentyl-phenylester shown in Example 3, in spite of no decrease in the clearing point, large decrease in viscosity at 5° C. could not be observed. From the foregoing, 4-hexyloxylbenzoic-4′-pentyl-phenylester having no bent core structure is presumed to have a molecular structure which does not contribute to the decrease in the viscosity.

### Embodiment 5

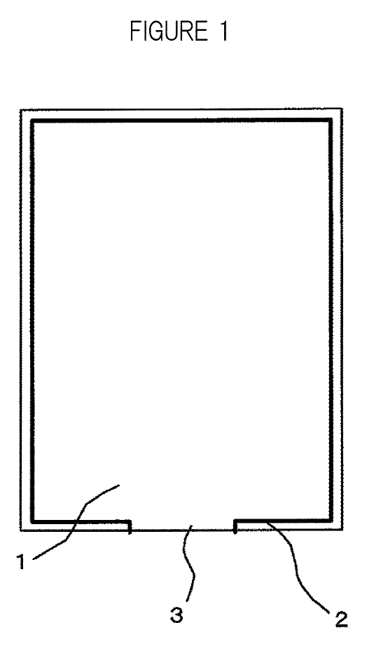

FIG. 1 shows an entire view of Liquid Crystal Device 1. The liquid crystal device has a size of 100 mm (long side)×100 mm (short side) and has a display portion of about 6-inch size across corners, and transparent glass substrates with polished surfaces each having a thickness of 1.1 mm were used. Common electrodes, signal electrodes, pixel electrodes, and the like were formed on the substrate, and an alignment layer was formed on the outmost surface of the substrate. Polyimide was used as the alignment layer in this embodiment. The polyimide was applied with a printer so that its thickness after baking might be approximately 0.07 to 0.1 μm. After that, the surfaces of the alignment layers were subjected to alignment treatment for orientation of liquid crystals. The alignment treatment was carried out with a rubbing machine by using rayon buff on rubbing rolls. Upper and lower substrates were adhered to each other as follows. A proper amount of polymer beads were mixed with a sealing agent 2 (an epoxy resin) and the resulting mixture was printed on the substrates so as to have the sealing agent 2, as shown in FIG. 1, by use of a seal mask. After that, the sealing agent was tentatively cured to combine the upper and lower substrates. Then, the sealing agent was cured while pressing the two substrates by use of a press. Inside the panel surface, spherical polymer beads were held between the substrates so that a size of the gap between them might be 8.0 μm in a state where liquid crystals were enclosed in the gap. A rubbing angle of the liquid crystal body was 45° with respect to the short side, and the direction of rubbing on the upper substrate was perpendicular to that on the lower substrate. A width of an opening for injecting liquid crystals 3 was 10 mm. The obtained device was a TN type liquid crystal device.

Next, a liquid crystal filling process for the liquid crystal device was described. The liquid crystal device was arranged, through not illustrated, in a vacuum container with the opening for injecting liquid crystals 3 being downward. Liquid crystals were in a liquid crystal bowl connected to a vertical-driving unit which was provided outside the vacuum container. In this example, Liquid Crystal Composition EXAMPLE 3 in which 5 wt % 4-hexyloxylbenzoic-3′-pentyl-phenylester represented by the molecular structure (20) had been added in ZLI-1132 (manufactured by Merck KGaA) was in the bowl. A constitution of the liquid crystal composition is already shown in Table 1. In addition, a little amount of a chiral compound is added in the liquid crystal composition. Hereinafter, this nematic liquid crystal composition is referred to as Nematic Liquid Crystal Composition A. The liquid crystals were put in a raised state in the liquid crystal bowl. Piping connected with a vacuum pump and a Pirani gage was provided outside the vacuum container. The vacuum pump was driven and a displacement volume was adjusted with an adjusting valve while the Pirani gage is monitored. Then, the vacuum container was exhausted for 120 minutes until vacuum degree reached 5 Pa. Next, the vertical-driving unit was driven, whereby the liquid crystals were dipped in the opening for injecting liquid crystals 3. After that, the adjusting valve was closed and an adjusting valve of leak piping was opened, and then nitrogen or air was introduced into the liquid crystal device. Next, Nematic Liquid Crystal Composition A was filled in the liquid crystal device. After the filling of the liquid crystals, the opening for injecting liquid crystals 3 was sealed with a ultraviolet curable agent (acrylic resin).

In this example, property values of Liquid Crystal Composition EXAMPLE 3 in which 5 wt % 4-hexyloxybeonzoic-3′-phenylester represented by the molecular structure (20) had been added in ZLI-1132 (manufactured by Merck KGaA) were almost the same as those of ZLI-1132 alone. A dielectric anisotropy was 10.3, a refractive index anisotropy was 0.14, and a viscosity at 25° C. is 20 cP. The most highlighted point in this example was fall response speed at 5° C. In general, whether or not the TN type liquid crystal device is good depends on fall response time. The fall response speed at 25° C. was 40 ms, which was not largely different from that of ZLI-1132 alone, but the fall response speed at 5° C. was 120 ms, which was remarkably improved compared to 140 ms of that of ZLI-1132 alone. In those evaluations, an applied voltage was 6 V. In addition, though there was possibility of decrease in the clearing point, the clearing point of Liquid Crystal Composition EXAMPLE 3 in the liquid crystal device was around 69° C., which was no problematic in practical use.

An operative temperature of the liquid crystal device of the present invention is −10° C. to 69° C. The operative temperature range is a usable temperature range of the liquid crystal device. A crystallization temperature of the liquid crystal composition ZLI-1132 used in the present invention is −20° C., and operation of the liquid crystal device of the present invention is also sufficiently secured even at low temperatures. In general, identification of the crystallization temperature is difficult and also needs much time, and therefore, shortening of evaluation time has been problematic. However, the crystallization temperature can be easily identified with the following means. That is, the liquid crystal composition incorporated in the liquid crystal device is kept at two different temperatures lower than a temperature of a low temperature test for a certain time. The device is kept at the lower temperature of those different two temperatures for a certain time, and then the device is kept at the higher temperature of those different two temperatures for a certain time, whereby the crystallization temperature can be easily identified. In this case, the temperature of the low temperature test is −10° C. The lower temperature of those different two temperatures is preferably a little higher than a glass transition temperature of the liquid crystal composition. This is because the temperature is effective for promoting crystal growth. The other higher temperature may be between the glass transition temperature and the temperature of the low temperature test, and is preferably between −60° C. to −40° C. This is because the temperature is effective for promoting crystal growth. Due to the two effects, the evaluation can be effectively conducted for a short time. It is convenient to use a thermal analysis device for those evaluation tests because the crystallization temperature can be detected as an endothermic peak. Note that the evaluations can be sufficiently conducted using a freezer, and the crystallization can be easily judged by visual observation.

In this example, improvement of the response speed of the TN type liquid crystal device at low temperatures was described. However, a reason of the improvement lies in decrease in the viscosity of the liquid crystal composition, and therefore, it is needless to say that the response speed at low temperatures can also be improved in liquid crystal devices of STN, IPS, VA, OCB, and the like.

### COMPARATIVE EXAMPLE 2

In this comparative example, a liquid crystal device was produced in the same way as in Example 5 except that Liquid Crystal Composition Comparative Example 1 in which 5 wt % 4-hexyloxybenzoic-4′-pentyl-phenyl ester represented by the molecular structure (22) had been added to ZLI-1132 (manufactured by Merck KGaA) was used as a liquid crystal composition. In addition, a little amount of a chiral compound was added to the liquid crystal composition. Hereinafter, this nematic crystal composition is referred to as Nematic Liquid Crystal Composition B.

Property values of Nematic Crystal Composition B were almost the same as ZLI-1132 alone, and a dielectric anistropy was 10.3, a refractive index anisotropy was 0.14, and a viscosity at 25° C. was 23 cP. A fall response time at 25° C. was 40 ms, which was not largely different from that of ZLI-1132 alone, but a fall response time at 5° C. was 170 ms, which was remarkably slow compared to 140 ms of that of ZLI-1132 alone.

While there have been described what are at present considered to be certain embodiments of the invention, it will be understood that various modifications may be made thereto, and it is intended that the appended claims cover all such modifications as fall within the true spirit and scope of the invention.

1. A liquid crystal composition comprising a compound represented by the following general formula (1), 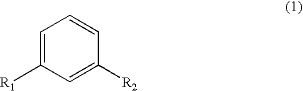
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

2. A liquid crystal composition comprising a compound represented by the following general formula (2), 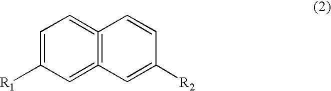
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

3. A liquid crystal composition comprising a compound represented by the following general formula (3), 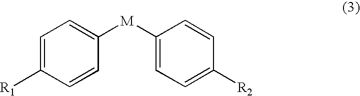
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent; and M is selected from CH₂, O, NH, and C═O.

4. A liquid crystal composition according to claim 1, comprising a compound represented by the following general formula (4), 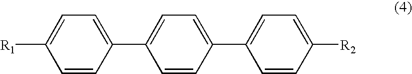
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

5. A liquid crystal composition according to claim 1, comprising a compound represented by the following general formula (5), 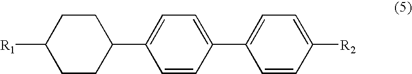
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

6. A liquid crystal composition according to claim 1, comprising a compound represented by the following general formula (6), 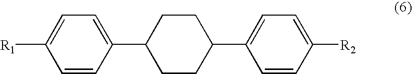
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

7. A liquid crystal composition according to claim 1, comprising a compound represented by the following general formula (7), 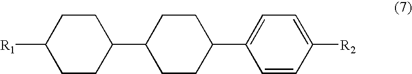
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

8. A liquid crystal composition according to claim 1, comprising a compound represented by the following general formula (8), 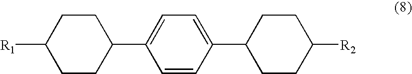
wherein: R₁ and R₂ are each independently selected from alkyl groups, alkoxy groups, F atom, Cl atom, OCF₃, CF₃, phenyl groups each having a substituent, and phenyl rings each connected with a bridging group and having a substituent.

9. A liquid crystal composition comprising a compound containing, in the same compound, two or more linking groups out of linking groups represented by the following general formulae (9), (10), and (11), 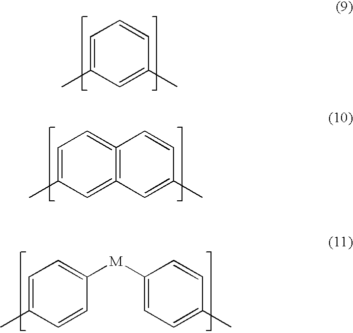
wherein M is selected from CH₂, O, NH, and C═O in the general formula (11).

10. A liquid crystal device comprising the compound represented by the general formula (1) in a liquid crystal composition according to claim 1.

11. A liquid crystal device comprising the compound represented by the general formula (2) in a liquid crystal composition according to claim 2.

12. A liquid crystal device comprising the compound represented by the general formula (3) in a liquid crystal composition according to claim 3.

13. A liquid crystal device comprising the compound represented by the general formula (4) in the liquid crystal composition according to claim 4.

14. A liquid crystal device comprising the compound represented by the general formula (5) in the liquid crystal composition according to claim 5.

15. A liquid crystal device comprising the compound represented by the general formula (6) in the liquid crystal composition according to claim 6.

16. A liquid crystal device comprising the compound represented by the general formula (7) in the liquid crystal composition according to claim 7.

17. A liquid crystal device comprising the compound represented by the general formula (8) in the liquid crystal composition according to claim 8.

18. A liquid crystal device comprising, in a crystal liquid composition according to claim 9, a compound containing two or more linking groups out of the linking groups represented by the general formulae (9), (10), and (11) in the same compound.
